(19) United States
(12) Patent Application Publication
(10) Pub. No.: US 2017/0313584 A1
(43) Pub. Date: Nov. 2, 2017

(54) CONVERSION OF METHANE AND ETHANE TO SYNGAS AND ETHYLENE

(71) Applicant: SABIC Global Technologies B.V., Bergen op Zoom (NL)

(72) Inventor: Aghaddin MAMEDOV, Sugar Land, TX (US)

(21) Appl. No.: 15/520,579

(22) PCT Filed: Oct. 23, 2015
(86) PCT No.: PCT/US2015/057053

§ 371 Date: Apr. 20, 2017

Related U.S. Application Data
(60) Provisional application No. 62/073,301, filed on Oct. 31, 2014.

(51) Int. Cl. C01B 3/40; B01J 23/34; C07C 2/84; C07C 5/48

(52) U.S. Cl. CPC C01B 3/40; C07C 5/48; B01J 23/34; C07C 2/84; C07C 2521/08; C01B 2203/1247; C01B 2203/1241; C01B 2203/1082; C01B 2203/061; C01B 2203/0495; C01B 2203/048; C01B 2203/0475; C01B 2203/0244; C07C 2523/04; C07C 2523/10; C07C 2523/34; C07C 2523/26

(57) ABSTRACT

Processes for conversion of methane and ethane into syngas and ethylene are provided. An exemplary process can include providing a reaction mixture of methane, ethane, oxygen, and carbon dioxide and contacting the reaction mixture with a catalyst that includes at least one metal oxide. The processes can be combined processes in which oxidative dry reforming of methane and dehydrogenation of ethane to ethylene with carbon dioxide and oxygen occur concurrently.

### FIELD

The presently disclosed subject matter relates to processes and systems for conversion of methane and ethane into syngas and ethylene.

### BACKGROUND

Synthesis gas, also known as syngas, is a gas mixture containing hydrogen (H₂) and carbon monoxide (CO). Syngas can also include carbon dioxide (CO₂). Syngas is a chemical feedstock that can be used in numerous applications. For example, syngas can be used to prepare liquid hydrocarbons, including olefins, via the Fischer-Tropsch process. Syngas can also be used to prepare methanol.

Ethylene (C₂H₄) is another chemical feedstock with numerous industrial uses. Ethylene is widely used as feedstock in polymerizations (e.g., for preparation of polyethylene) and in oligomerizations to generate higher olefins and other compounds. Ethylene is also used to prepare ethylene oxide, halogenated compounds, ethylbenzene, and many other compounds.

Syngas is commonly generated on large scale from methane (CH₄), e.g., through steam reforming processes. Ethylene is produced on large scale from ethane (C₂H₆), e.g., through steam cracking. These existing processes can suffer from drawbacks. For example, steam cracking and steam reforming processes can be affected by harmful coke formation. Steam cracking and steam reforming processes can also be highly endothermic and energy intensive.

One alternative route to converting ethane to ethylene can be dry dehydrogenation of ethane in the presence of carbon dioxide, oxygen, and a catalyst. However, catalysts used for dry dehydrogenation of ethane can be incompatible with methane. Accordingly, rather than using combined mixtures of methane and ethane, methane and ethane may need to be separated, and purified ethane can then be dehydrogenated to ethylene. Separation of methane and ethane can be costly.

Shale gas is a rich source of both methane and ethane. Shale gas is a form of natural gas that can include methane, ethane, higher hydrocarbons (e.g., propane and butane), carbon dioxide, nitrogen (N₂), and hydrogen sulfide (HS). Depending on the source of the shale gas, the composition may vary.

Thus, there remains a need for improved processes for preparation of syngas and ethylene from methane and ethane that do not require prior separation of methane and ethane but can instead be performed with combined mixtures (e.g., shale gas).

### SUMMARY OF THE DISCLOSED SUBJECT MATTER

The presently disclosed subject matter provides processes for conversion of methane and ethane into syngas and ethylene.

In one embodiment, an exemplary process for conversion of methane and ethane into syngas and ethylene can include providing a reaction mixture that includes methane, ethane, oxygen, and carbon dioxide. The process can further include contacting the reaction mixture with a catalyst that includes at least one metal oxide such as chromium oxides, manganese oxides, copper oxides, tin oxides, lanthanum oxides, cerium oxides, and tungsten oxides, to provide a product mixture including syngas and ethylene.

In certain embodiments, the reaction mixture can include shale gas. In certain embodiments, the reaction mixture can be dry.

In certain embodiments, the catalyst can include a solid support. In certain embodiments, the solid support can include at least one support such as alumina, silica, and magnesia. In certain embodiments, the catalyst can include the metal oxide in an amount between about 5% and about 15%, by weight, relative to the total weight of the catalyst. The catalyst can include the metal oxide in an amount of about 15%, by weight, relative to the total weight of the catalyst.

In certain embodiments, the catalyst can include a basic metal oxide. In certain embodiments, the basic metal oxide can include at least one of lithium oxides, sodium oxides, potassium oxides, calcium oxides, strontium oxides, barium oxides, and lanthanum oxides. In certain embodiments, the basic metal oxide can include at least one of lithium oxides, sodium oxides, and potassium oxides. In certain embodiments, the catalyst can include the basic metal oxide in amount between about 1% and about 5%, by weight, relative to the total weight of the catalyst. The catalyst can include the basic metal oxide in an amount between about 1% and about 1.5%, by weight, relative to the total weight of the catalyst.

In certain embodiments, the reaction mixture can be contacted with the catalyst at a temperature between about 650° C. and about 950° C. In certain embodiments, the reaction mixture can be contacted with the catalyst at a temperature between about 800° C. and about 850° C.

In certain embodiments, the process can include separating water from the product mixture. In certain embodiments, separating water from the product mixture can include cooling the product mixture.

In certain embodiments, the process can include separating syngas and ethylene from the product mixture to provide purified syngas and purified ethylene. In certain embodiments, the process can include converting purified syngas into methanol.

In one embodiment, an exemplary process for conversion of shale gas into syngas and ethylene can include providing shale gas that includes methane and ethane and mixing the shale gas with oxygen and carbon dioxide to provide a reaction mixture. The process can further include contacting the reaction mixture with a catalyst.

The catalyst can include a solid support such as alumina, silica, and magnesia. The catalyst can further include at least one of chromium oxides, manganese oxides, tin oxide, lanthanum oxides, cerium oxides, and tungsten oxides, in an amount between about 5% to about 15%, by weight, relative to the total weight of the catalyst. The catalyst can further include at least one of lithium oxides, sodium oxides, and potassium oxides, in an amount between about 1% to about 5%, by weight, relative to the total weight of the catalyst.

### BRIEF DESCRIPTION OF THE DRAWINGS

FIG. 1 is a schematic diagram showing an exemplary system that can be used in conjunction with processes for conversion of methane and ethane into syngas and ethylene.

FIG. 2 is a schematic diagram showing another exemplary system that can be used in conjunction with processes for conversion of methane and ethane into syngas and ethylene.

### DETAILED DESCRIPTION

The presently disclosed subject matter provides processes for conversion of shale gas and other methane/ethane mixtures to syngas and ethylene without requiring initial separation of methane and ethane. The processes can include two distinct reactions—oxidative dry reforming of methane to syngas and dehydrogenation of ethane to ethylene—occurring concurrently and promoted by a single catalyst. The processes of the presently disclosed subject matter can have advantages over existing processes, including reduced cost, increased energy efficiency, and improved control of reaction temperature, as described below.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

Oxidative dry reforming of methane is a process in which methane is reacted with carbon dioxide and oxygen to provide carbon monoxide, hydrogen, and water. Oxidative dry reforming can be summarized by the following chemical equation:

2CH₄+CO₂+O₂→3CO+3H₂+H₂O (1)

Oxidative dry reforming can accordingly generate syngas with a hydrogen:carbon monoxide ratio of approximately 1:1.

Dehydrogenation of ethane to ethylene with carbon dioxide and oxygen provides a mixture of ethylene, carbon monoxide, and water. Dehydrogenation of ethane to ethylene with carbon dioxide and oxygen can be summarized by the following chemical equation:

2C₂H₆+CO₂+½O₂→2C₂H₄+CO+2H₂O (2)

Combining oxidative dry reforming of methane and dehydrogenation of ethane to ethylene with carbon dioxide and oxygen can provide a combined process. The overall balanced chemical equation for the combined process can be summarized as follows:

2C₂H₆+2CH₄+2CO₂+1.5O₂→2C₂H₄+4CO+3H₂+2H₂O (3)

While the chemical equations above depict conversion of methane to syngas and conversion of ethane to ethylene, it should be understood that methane can also be converted to ethylene. In certain embodiments of the presently disclosed subject matter, catalysts that promote oxidative dry reforming of methane and dehydrogenation of ethane to ethylene with carbon dioxide and oxygen can also promote conversion of methane to ethylene under the same reaction conditions.

Processes for conversion of methane and ethane into syngas and ethylene of the presently disclosed subject matter can generally include providing a reaction mixture that includes methane, ethane, oxygen, and carbon dioxide. The processes can further include contacting the reaction mixture with a catalyst that includes at least one metal oxide such as one or more chromium oxides, manganese oxides, copper oxides, tin oxides, lanthanum oxides, cerium oxides, and tungsten oxides, to provide a product mixture including syngas and ethylene.

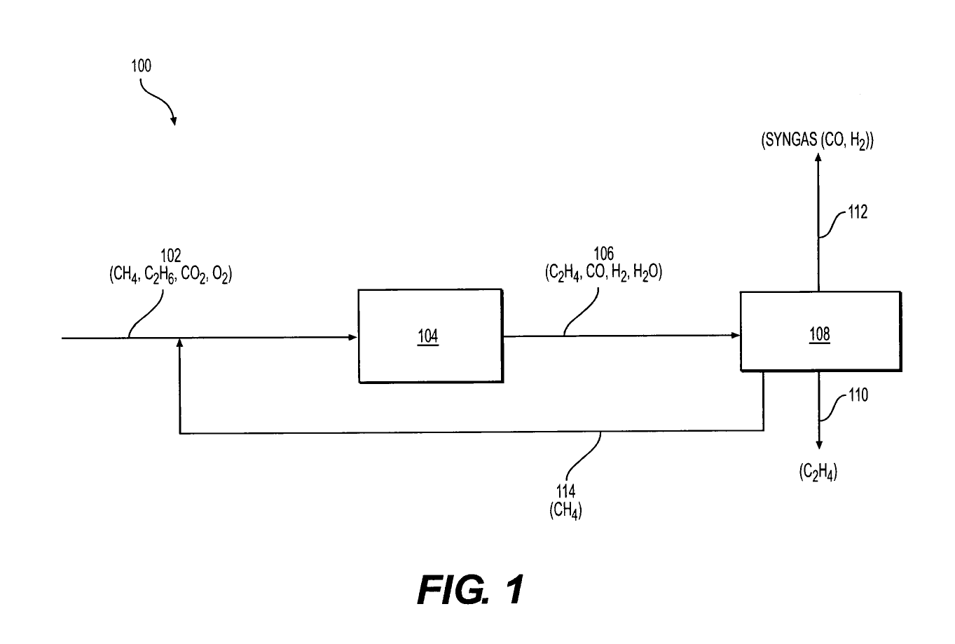

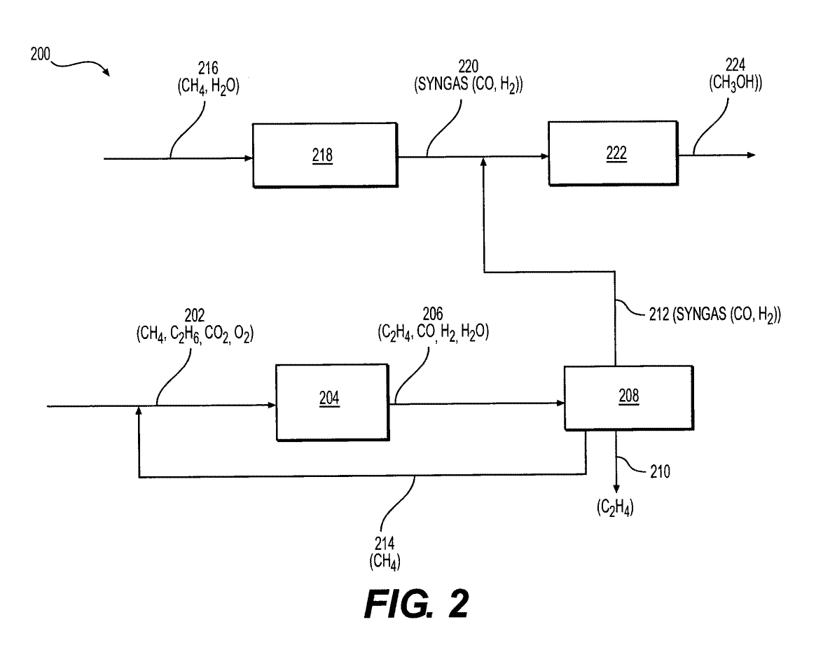

For the purpose of illustration and not limitation, FIGS. 1 and 2 are schematic representations of exemplary systems that can be used in conjunction with the processes of the presently disclosed subject matter. The system 100, 200 can include a reaction mixture stream 102, 202 that includes methane, ethane, oxygen, and carbon dioxide. The proportions of methane, ethane, oxygen, and carbon dioxide in the reaction mixture stream 102, 202 can be varied. In certain embodiments, the ratio of ethane:methane:carbon dioxide:oxygen can be about 2:2:2:1.5. In certain embodiments, an excess of methane can be used. When an excess of methane is used, the amount of oxygen can be varied.

In certain embodiments, the reaction mixture stream 102, 202 can include shale gas. That is, at least a portion of the methane and/or ethane in the reaction mixture stream 102, 202 can be derived from shale gas. In certain embodiments, at least a portion of the carbon dioxide in the reaction mixture stream 102, 202 can also be derived from shale gas. In certain embodiments, all of the reaction mixture stream 102, 202 can be derived directly from shale gas. In certain embodiments, the reaction mixture stream 102, 202 can be a mixture of methane and ethane from which hydrogen sulfide has been removed by desulfurization.

In certain embodiments, the reaction mixture stream 102, 202 can be dry. That is, the reaction mixture stream 102, 202 can be free of water.

The reaction mixture stream 102, 202 can be fed to a reactor 104, 204. The reactor 104, 204 can be of various designs known in the art. In certain embodiments, the reactor can be a fixed bed plug flow reactor. In certain embodiments, the reactor can be a fluidized bed or riser-type reactor. In certain embodiments, the reactor can be a quartz reactor or metal reactor.

The reactor 104, 204 can include a catalyst. On feeding the reaction mixture 102, 202 into the reactor 104, 204, the reaction mixture can come into contact with the catalyst and react to provide a product mixture that includes syngas (carbon monoxide and hydrogen) and ethylene.

The catalyst can include one or more metal oxides. By way of non-limiting example, suitable metal oxides can include chromium oxides (e.g., Cr₂O₃), manganese oxides (e.g., MnO, MnO₂, Mn₂O₃, or Mn₂O₇), copper oxides (e.g., CuO), tin oxides (e.g., SnO₂), lanthanum oxides (e.g., La₂O₃), cerium oxides (e.g., CeO₂), and tungsten oxides (e.g., WO₃). In certain embodiments, acidic metal oxides can cause over-oxidation of ethane (e.g., to carbon monoxide and/or carbon dioxide). In certain embodiments, the catalyst can include oxides of two, three, four, or more different metals (elements). By way of non-limiting example, the catalyst can include a first oxide selected from one or more of oxides of Mn, W, Sn, and La and a second oxide selected from one or more oxides of Ce, Cu, and Cr.

In certain embodiments, the catalyst in the reactor 104, 204 can be used as a bulk mixture of oxides. By way of non-limiting example, the reactor 104, 204 can be packed with particles, granules, and/or pellets of catalyst.

In certain embodiments, the catalyst in the reactor 104, 204 can include a solid support. That is, the catalyst can be solid-supported. In certain embodiments, the solid support can include various metal salts, metalloid oxides, and metal oxides, e.g., titania (titanium oxide), zirconia (zirconium oxide), silica (silicon oxide), alumina (aluminum oxide), magnesia (magnesium oxide), and magnesium chloride. In certain embodiments, the solid support can include alumina (Al₂O₃), silica (SiO₂), magnesia (MgO), or a combination thereof.

In certain embodiments, the catalyst can include one or more metal oxides in an amount between about 5% and about 15%, by weight, relative to the total weight of the catalyst. For example, when the catalyst includes a solid support, the catalyst can include the metal oxide in an amount between about 5% and about 15%, by weight, relative to the total weight of the catalyst, and the remainder of the catalyst can be solid support. The catalyst can include the metal oxide in an amount of about 15%, by weight, relative to the total weight of the catalyst. Catalyst loading and metal oxide loading can be proportional to reactor size. By way of non-limiting example, a quartz or metal reactor having an internal diameter of 2.5 cm and a length of 45 cm can be loaded with an amount of catalyst between about 0.5 mL and about 3 mL, e.g., between about 0.5 mL and about 1.5 mL.

In certain embodiments, the catalyst can include a basic metal oxide. Basic metal oxides are metal oxides with basic properties. For example, basic metal oxides include metal oxides that can react with an acid to form a salt and water. In certain embodiments, the basic metal oxide can include at least one basic metal oxide such as lithium oxides (e.g., Li₂O), sodium oxides (e.g., Na₂O), potassium oxides (e.g., K₂O), calcium oxides (e.g., CaO), strontium oxides (e.g., SrO), barium oxides (e.g., BaO), and lanthanum oxides (e.g., La₂O₃). In certain embodiments, the basic metal oxide can be Li₂O, Na₂O, or K₂O.

In certain embodiments, the catalyst can include one or more basic metal oxides in an amount between about 1% and about 5%, by weight, relative to the total weight of the catalyst. For example, when the catalyst includes a solid support and one or more additional metal oxides, the catalyst can include the basic metal oxide in an amount between about 1% and about 5%, by weight, relative to the total weight of the catalyst, and the remainder of the catalyst can be solid support and the one or more additional metal oxides. In certain embodiments, the catalyst can include the basic metal oxide in an amount between about 1% and about 1.5%, by weight, relative to the total weight of the catalyst.

In certain embodiments wherein the catalyst includes one or more basic metal oxides, the catalyst can combine both basic and redox properties. For example, the catalyst can include both a transition metal or lanthanide oxide (e.g., an oxide of Mn or Cr) capable of oxidation and reduction to different oxidation states as well as a basic oxide (e.g., an oxide of K or Na). Individual transition metal oxides and lanthanide oxides can have both basic and redox character (e.g., La₂O₃).

In certain embodiments, the metal oxides (including basic oxides) of the catalysts of the presently disclosed subject matter can be prepared by precipitation. In certain embodiments, metal oxides can be precipitated from corresponding nitrate salts by treatment with NH₄OH. In certain embodiments wherein the catalyst includes oxides of more than one metal, the metal oxides can be co-precipitated by treatment of nitrate salts of the corresponding metals with NH₄OH. By way of non-limiting example, metal oxides can be precipitated or co-precipitated by treatment of the corresponding nitrate salts with NH₄OH, followed by washing, drying at 120° C., and calcination at 700° C. for 4 hours.

In certain embodiments, the reaction mixture can be contacted with the catalyst at a temperature between about 650° C. and about 950° C. That is, the temperature in the reactor 104, 204 can be between about 650° C. and about 950° C. In certain embodiments, the reaction mixture can be contacted with the catalyst at a temperature between about 800° C. and about 850° C.

In certain embodiments, the reactor 104, 204 can have a gas hourly space velocity (GHSV) of between about 2,000 h⁻¹ and about 20,000 h⁻¹, e.g., between about 5,000 h⁻¹ and about 10,000 h⁻¹. By way of non-limiting example, the GHSV of the reactor 104, 204 can be about 7,200 h⁻¹. In certain embodiments, the reaction mixture 102, 202 can have a contact time of between about 0.1 seconds and about 5 seconds. By way of non-limiting example, the reaction mixture 102, 202 can have a contact time of about 0.5 seconds.

In certain embodiments, the reactor 104, 204 can be operated at atmospheric pressure. In other embodiments, the reactor 104, 204 can be operated at elevated pressure. For example, the reactor 104, 204 can be operated at a pressure between atmospheric pressure and about 30 bar, e.g., in a range between about 20 bar and about 25 bar.

A product mixture stream 106, 206 can be removed from the reactor 104, 204. The product mixture stream 106, 206 can include ethylene, carbon monoxide, hydrogen, and water. That is, the product mixture stream can include ethylene, syngas (carbon monoxide and hydrogen), and water. In certain embodiments, the product mixture stream can also contain unreacted methane and/or ethane.

The product mixture stream 106, 206 can be fed to a separation unit 108, 208. The separation unit can separate and remove water from the product mixture. In certain embodiments, separating water from the product mixture can include cooling the product mixture. In other words, the separation unit 108, 208 can cool the product mixture to condense water. By way of non-limiting example, the temperature within the separation unit 108, 208 can be between about 5° C. and about 10° C. and the pressure can be between about 1 bar and 20 bar.

In certain embodiments, the separation unit 108, 208 can separate syngas (carbon monoxide and hydrogen) and ethylene from the product mixture. The separation unit 108, 208 can separate various components by distillation. A purified ethylene stream 110, 210 and a purified syngas stream 112, 212 can be removed from the separation unit 108, 208. Ethylene and syngas can be isolated as products of the process. In certain embodiments, a methane stream 114, 214 can also be removed from the separation unit. The methane stream 114, 214 can be fed into the reaction mixture stream 102, 202. In this way, unreacted methane can be recycled through the process. Unreacted ethane can also be removed from the separation unit 108, 208 and recycled.

The conversion of methane and ethane in the processes and systems of the presently disclosed subject matter can vary. By way of non-limiting example, the conversion of methane can be in a range from about 5% to about 95%, e.g., in a range from about 10% to about 50% or in a range from about 25% to about 35%. By way of non-limiting example, the conversion of ethane can be in a range from about 5% to about 95%, e.g., in a range from about 50% to about 90% or in a range from about 60% to about 75%.

In certain embodiments, the system 200 can include a steam reforming reactor 218 and a methanol reactor 222. In certain embodiments, the syngas stream 212 separated from the separation unit 208 can be fed into a methanol reactor 222. That is, processes for conversion of methane and ethane into syngas and ethylene can further include converting purified syngas into methanol. A steam reforming mixture stream 216 that includes methane and water can be fed to the steam reforming reactor 218. Steam reforming of methane can occur within the reactor 218 under conditions known in the art to provide a steam reforming product stream 220 that includes syngas (carbon monoxide and hydrogen). The steam reforming product stream 220 and the syngas stream 212 derived from the separation unit 208 can be combined and fed together to the methanol reactor 222. The methanol reactor 222 can convert syngas to methanol under conditions known in the art. A methanol stream 224 can be removed from the methanol reactor 222.

Steam reforming of methane can provide syngas with a hydrogen:carbon monoxide ratio of about 3:1 (mole:mole). As described above, oxidative dry reforming can generate syngas with a hydrogen:carbon monoxide ratio of approximately 1:1 (mole:mole). The syngas stream 212 derived from the separation unit 208 can be further enriched in carbon monoxide derived from dehydrogenation of ethane, such that the molar ratio of hydrogen:carbon monoxide in the syngas stream 212 can be less than 1:1, about 1:1, or above 1:1 but less than 2:1. Consequently, mixing the stream reforming product stream 220 with the syngas stream 212 derived from the separation unit 208 in various proportions can provide a syngas mixture with hydrogen:carbon monoxide ratios between about 3:1 and about 1:1, e.g., about 2:1. Syngas with a hydrogen:carbon monoxide ratio 2:1 can be used to prepare methanol.

The processes of the presently disclosed subject matter can have advantages over existing processes for conversion of methane and ethane into syngas and ethylene. Because the reaction mixture stream 102, 202 can be a dry mixture of methane, ethane, carbon dioxide, and oxygen (i.e., free of water), the processes of the present disclosure can be free of coke formation. That is, there can be no coke formation in the reactor 104, 204 or in downstream equipment. An absence of coke formation obviates the need for costly and inefficient regeneration of catalysts due to buildup of coke.

An additional advantage of the presently disclosed subject matter can be the use of oxidative dry reforming for conversion of methane to syngas, rather than use of steam reforming or oxidative reforming with oxygen (in the absence of carbon dioxide). Whereas steam reforming is highly endothermic (and consequently highly energy intensive) and oxidative reforming with oxygen is highly exothermic (and consequently able to cause problematic exotherms), oxidative dry reforming is only mildly exothermic, which can reduce energy consumption and facilitate control of heat released by the reaction.

### EXAMPLES

### Example 1

0.5 mL of a K—Ce—Mn—Cr/SiO₂ catalyst was loaded into a quartz reactor with an internal diameter (ID) of 2.5 cm and a length of 45 cm. The K—Ce—Mn—Cr/SiO₂ catalyst had the following metal oxide composition: 1.5% K, 3% Ce, 10% Mn, and 4% Cr, with the balance being oxygen. The reactor was located in a heated furnace. The reactor was heated to 850° C., and a reaction mixture stream containing 60 mol % CH₄, 12 mol % C₂H₆, 16 mol % CO₂, and 12 mol % O₂ was fed to the reactor at a flow rate of 40 cc/minute. A product mixture stream was removed from the reactor. The conversion of methane was 25%, and the conversion of ethane was 70%. The content of CO in the product mixture was about 8-9 mol % and the content of H₂ in the product mixture was about 7-8 mol %, with the remainder consisting primarily of ethylene, ethane, methane, and CO₂. The components of the product mixture were then separated by distillation. Hydrocarbons (including ethylene, ethane, and methane) could be obtained as purified individual compounds after separation. After separation, approximately half of the CO in the product mixture was fed into a methanol reactor. The remainder of the CO in the product mixture was mixed with the H₂ in the product mixture to form a syngas mixture having a H₂:CO ratio of approximately 2:1.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

What is claimed is:

1. A process for conversion of methane and ethane into syngas and ethylene, comprising:
a. providing a reaction mixture comprising methane, ethane, oxygen, and carbon dioxide;
b. contacting the reaction mixture with a catalyst comprising at least one metal oxide selected from the group consisting of chromium oxides, manganese oxides, copper oxides, tin oxides, lanthanum oxides, cerium oxides, and tungsten oxides, to provide a product mixture comprising syngas and ethylene.

2. The process of claim 1, wherein the reaction mixture comprises shale gas.

3. The process of claim 1, wherein the reaction mixture is dry.

4. The process of claim 1, wherein the catalyst further comprises a solid support.

5. The process of claim 4, wherein the solid support comprises at least one support selected from the group consisting of alumina, silica, and magnesia.

6. The process of claim 4, wherein the catalyst comprises the metal oxide in an amount between about 5% and about 15%, by weight, relative to the total weight of the catalyst.

7. The process of claim 6, wherein the catalyst comprises the metal oxide in an amount of about 15%, by weight, relative to the total weight of the catalyst.

8. The process of claim 1, wherein the catalyst further comprises a basic metal oxide.

9. The process of claim 8, wherein the basic metal oxide comprises at least one basic metal oxide selected from the group consisting of lithium oxides, sodium oxides, potassium oxides, calcium oxides, strontium oxides, barium oxides, and lanthanum oxides.

10. The process of claim 9, wherein the basic metal oxide comprises at least one basic metal oxide selected from the group consisting of lithium oxides, sodium oxides, and potassium oxides.

11. The process of claim 8, wherein the catalyst comprises the basic metal oxide in an amount between about 1% and about 5%, by weight, relative to the total weight of the catalyst.

12. The process of claim 10, wherein the catalyst comprises the basic metal oxide in an amount between about 1% and about 1.5%, by weight, relative to the total weight of the catalyst.

13. The process of claim 1, wherein the reaction mixture is contacted with the catalyst at a temperature between about 650° C. and about 950° C.

14. The process of claim 13, wherein the reaction mixture is contacted with the catalyst at a temperature between about 800° C. and about 850° C.

15. The process of claim 1, further comprising separating water from the product mixture.

16. The process of claim 15, wherein separating water from the product mixture comprises cooling the product mixture.

17. The process of claim 1, further comprising separating syngas and ethylene from the product mixture to provide purified syngas and purified ethylene.

18. The process of claim 17, further comprising converting purified syngas into methanol.

19. A process for conversion of shale gas into syngas and ethylene, comprising:
a. providing shale gas comprising methane and ethane;
b. mixing the shale gas with oxygen and carbon dioxide to provide a reaction mixture;
c. contacting the reaction mixture with a catalyst, wherein the catalyst comprises:
i. a solid support selected from the group consisting of alumina, silica, and magnesia;
ii. at least one metal oxide selected from the group consisting of chromium oxides, manganese oxides, tin oxides, lanthanum oxides, cerium oxides, and tungsten oxides, in an amount between about 5% to about 15%, by weight, relative to the total weight of the catalyst;
iii. at least one basic oxide selected from the group consisting of lithium oxides, sodium oxides, and potassium oxides, in an amount between about 1% to about 5%, by weight, relative to the total weight of the catalyst.
